(19) United States
(12) Patent Application Publication
(10) Pub. No.: US 2024/0122216 A1
(43) Pub. Date: Apr. 18, 2024

(54) A FOOD PROCESSING LINE AND METHOD FOR CONTROLLING A FOOD PROCESSING LINE

(71) Applicant: MAREL FURTHER PROCESSING B.V., Boxmeer (NL)

(72) Inventors: Hendrikus Cornelis Koos VAN DOORN, Boxmeer (NL); Bernardus Wilhelmus Franciscus LEFERINK, Boxmeer (NL); Johannes Martinus MEULENDIJKS, Boxmeer (NL)

(21) Appl. No.: 18/263,853

(22) PCT Filed: Feb. 7, 2022
(86) PCT No.: PCT/EP2022/052885

§ 371 Date: Aug. 1, 2023

(30) Foreign Application Priority Data
Feb. 8, 2021 (NL) 2027519

(51) Int. Cl. A23L 5/00; G05B 19/418

(52) U.S. Cl. CPC A23L 5/00; G05B 19/4184; G05B 2219/32179

(57) ABSTRACT

The present invention relates to a processing line and a method for controlling a food processing line, the food processing line comprising a plurality of processing stations and at least one utility supply station. Further at least one food product sensor is provided, and at least one utility sensor and at least one processing sensor. A processing line controller is provided comprising a data collection module, input means for specifying at least one desired food product output characteristic, input means for specifying a nominal operating condition, an anomaly detection module configured to detect an anomaly, and a root cause module configured to determine a root cause of the detected anomaly. A corrective measure module is configured to determine a corrective measure in response to a detected anomaly and to provide the corrective measure to at least one physical actuator.

### FIELD OF THE INVENTION

The present invention relates to a food processing line and method for controlling a food processing line.

### BACKGROUND OF THE INVENTION

Food processing lines for processing a food product in a plurality of subsequent processing stations. These processing stations need to be controlled such that the food product undergoes all required processing operations in the right order and with the right magnitude in order to fulfil the requirements set for the end product. Known food processing lines compare the end product with the product requirements in order to determine whether the end product fulfils the requirements and/or whether the food processing line need to be controlled in order to have future end products fulfilling the requirements.

Modern day processing lines commonly process large quantities of food products and use large amounts of resources while processing them. A disadvantage of the known food processing lines is that when an end product is detected that does not fulfil its specifications, a large quantity of resources is already used for simultaneously processed products that in many cases do not fulfil their requirements either. In many cases this results in a loss of food processing resources and potential waste due to product rejection.

### SUMMARY OF THE INVENTION

In a first aspect of the invention, a food processing line for processing a food product is provided, comprising:
a plurality of processing stations in which the food product is subjected to one or more processing operations;
at least one utility supply station providing a processing utility to one or more of the processing stations;
at least one food product sensor configured to acquire a food product condition measure;
at least one utility sensor configured to acquire a utility condition measure;
at least one processing sensor configured to acquire a processing station condition measure;
a processing line controller for controlling the food processing line, comprising:
   a data collection module for collecting sensor information, configured to:
      receive sensor information from the at least one food product sensor, the at least one utility sensor and the at least one processing sensor;
      store sensor information on a storage means;
      communicate stored sensor information via an electronic communication line;
   input means for specifying at least one desired food product output characteristic;
   input means for specifying a nominal operating condition for the utility supply station and for the processing station;
   an anomaly detection module configured to in operation detect an anomaly from the nominal operating condition based on the collected sensor information;
   a root cause module configured to in operation determine a root cause of the detected anomaly using a statistical data analysis;
   a corrective measure module configured to in operation determine a corrective measure in response to a detected anomaly and to provide the corrective measure to at least one physical actuator in the food processing line in order to control the food processing line such that the food product is processed in accordance with the desired food product output characteristic.

Exemplary discrete food products processed in a food process line according to the invention are burgers such as hamburgers, burgers, balls, nuggets, schnitzels, sausages, etc.

The food processing line comprises a plurality of processing stations, which are commonly connected with each other by means of automated transport means on which the food product is transported from one processing station to the next, however the food product may also be transported manually by an operator from one processing station to the next. In or at a processing station the food product is subjected to one or more processing operations. Exemplary discrete food processing stations according to the invention include food preparation station for preparing a pumpable food mass, cold store for cold storing a pumpable food mass, pump station for pumping a pumpable food mass, forming station for forming discrete food products from a pumpable food mass, dry coater for coating the discrete food products with a dry coating, wet coating device for coating the discrete food products with a wet coating, optionally comprising a batter mixer, fryer for frying the discrete food products, a heater such as a fryer and/or an oven for subjecting the discrete food products to a heat treatment and/or a freezer for freezing the discrete food products.

The food processing line further comprises at least one utility supply station providing a processing utility to one or more of the processing stations. In the context of the present invention, a utility is a supply in aid of one or more food processing operations, not being a base material for the food product itself. A utility supply station may be inline or adjacent to the food processing stations, but may alternatively be physically remote from the processing stations. A utility supply station may be exclusively supplying its utility to a single processing station, or share its utility over multiple processing stations in a single food processing line, or even over multiple processing lines in a production facility. Exemplary utility supplies include thermal oil, steam, pressurized air, electricity, pressurized water, etc.

In the food processing line according to the invention, at least one food product sensor is provided, preferably a plurality of food product sensors throughout the food processing line. Such food product sensor may travel along with a selected food product through the whole or a part of its travel through the food processing line, or alternatively be provided at one or more locations in the processing line. Preferably a plurality of food product sensors is provided along a substantial part through the food processing line. The at least one food product sensor is communicatively connected to a processing line controller, either directly or via an interface. Such connection may be implemented physically or wirelessly. The sensor may provide its measurement e.g. at a regular interval, upon passing a certain threshold or upon request of a communication station. The at least one food product sensor is configured to acquire a food product condition, such as e.g. a core temperature of a food product, a surface temperature of a food product, weight of a discrete food product or a collection of discrete food products, a product surface color, a product dimension (e.g. width, length, height), a product appearance characteristic (e.g. shape, curvature, surface spots, color differences, etc). An exemplary and frequently applied sensor associated with a food processing line is a temperature sensor, e.g. using a thermocouple or a thermal radiation thermometer such as an infrared thermometer. Contact temperature sensor types are conceivable, but also non-contact sensor types. Other commonly applied sensors involve an optical system such as a camera, e.g. detecting volume, colour and one or more dimensions such as shape, width, length and/or height. Weight sensors such as scales are also commonly known. Camera modules may be equipped with additional image processing features at the camera or remotely in order to provide interpreted information to the system. Examples of such image processing may include stitching multiple images, image correction, color measurement, threshold operations, or more complex operations such as image interpretations using machine learning algorithms to identify (position and/or orientation of) individual products on a conveyor belt, for example algorithms like convolutional neural networks such as “You Only Look Once” (YOLO), Fast R-CNN, Masked R-CNN, Retina-Net, and/or Single-Shot MultiBox Detector (SSD), or machine learning algorithms for labelling individual products based on a specified product characteristic like shape, color, unintentionally merged products (marriages), etc. Known algorithms for this kind of labelling by interpreting the images include DenseNet and Masked R-CNN.

In the food processing line according to the invention, at least one utility sensor is provided, preferably at least one utility sensor for each utility supplying utility supply station. It will be appreciated by the skilled person that the type of utility supply sensor depends on the specifics of the utility supply station. Exemplary utility sensors include a temperature sensor and/or an oil flow sensor for a thermal oil heater station; a temperature and/or pressure sensor for a steam supply station; a pressure and/or flow sensor for a pressurized air supply station; an electrical current, voltage, power and/or signal condition sensor for an electricity supply station; a pressure, temperature and/or flow sensor for a pressurized water supply station, etc.

In the food processing line according to the invention, at least one processing sensor is provided, preferably at least one processing sensor for each food processing station in the food processing line. It will be appreciated by the skilled person that the type of processing sensor depends on the specifics of the processing station. Exemplary processing sensors include climate sensors configured to measure one or more aspects of the climate at or in a processing station, such as temperature and/or humidity, and sensors to determine a dwell time of the product inside a processing station.

Below exemplary embodiments of food processing stations are elucidated, with optional actuators and/or sensors. A food processing line may comprise these and/or other food processing stations. Although in some cases the character of a food product dictates the order of a subset of processing stations, some food processing facilities may be configured to flexibly connect one or more of these processing stations by configurable connecting food transport belts.

Food Preparation Station

A food preparation station for preparing a pumpable food mass e.g. comprises a tumbler, a cutter and/or a grinder. Possibly an additive addition device is provided for adding marinade and/or seasoning. Possibly a CO2 addition device is provided.

Exemplary actuators associated with such a food preparation station are speed actuators setting the speed of operation, e.g. of the tumbler, cutter, grinder. Another possible actuator is the additive (marinade, seasoning, CO2) addition rate. Preferably actuators are provided setting the ingredients of additive, e.g. marinade, in particular salt and glucose.

An exemplary sensor in a food preparation station measures the glucose content of food mass, and/or the density of the mass, and/or the viscosity.

Exemplary controllable parameters in a food preparation station include:
the marinade addition rate and/or the marinade ingredients in the food preparation station
the CO2-addition in the food preparation station
the temperature in the food preparation station, for example using an electric cooling and or heating means.

Cold Store for Pumpable Food Mass

In a cold store a food mass is allowed to reside, e.g. as a buffer or to actively bring the food mass to a desired temperature. In embodiments, liquid nitrogen cooling is performed.

Exemplary controllable parameters in a cold store for pumpable food mass include:
the temperature in the cold store, e.g. the supply valve of a liquid nitrogen supply

Pump Station for a Pumpable Food Mass

A pump station generally comprises a hopper and a pump. The pump may operate continuously such as a screw pump, or batch-wise such as a plunger pump. An exemplary actuator associated with such a pump station is a speed actuator setting the speed of operation of the pump. Also common is a thermal actuators to set the temperature in the pump. Such a pump station may comprise a viscosity sensor for the food mass.

Exemplary controllable parameters in a pump station for a pumpable food mass include
the temperature in the pump
the pump speed.

Forming Station for Forming Discrete Food Products

In a forming station, discrete food products are formed from a pumpable food mass. Such a station generally comprises a hopper for the food mass, a forming device and conveying means for the formed, discrete food products, such as a belt conveyor. The forming device e.g. comprises a moulding device or a sausage machine.

It is possible that the pump station and the forming station are separate stations, e.g. produced by different machine factories. However, in embodiments, the forming station is provided with an associated pump, and possibly also a hopper. This ensemble of pump and forming station can be produced as a single unit by the same machine factory.

Such a moulding device is commercially available from the same applicant. It is described e.g. in multiple applications of the same applicant, e.g. WO0030458, WO2004002229, WO2005107481, WO2010110655, WO2014017916 etc. etc. Exemplary actuators of such a moulding device include actuators setting the fill pressure, fill plate pressure settings, thermal actuators for heating of the hopper and/or the piping between hopper and moulding device, pump speed, etc.

Sausage machines are also commercially available from the same applicant. Exemplary actuators of such a sausage machine include thermal actuators for heating of the hopper and/or the piping between hopper and sausage machine, pump speed, etc.

Commonly temperature sensors are applied to measure the temperature of the food in the hopper and in the forming device, and scales to measure the weight.

Exemplary controllable parameters in a forming station include:
the fill pressure and/or the fill plate pressure settings of the moulding device
the fill pressure and/or the fill plate pressure settings
the temperature of the piping between hopper and moulding device/sausage machine
the operation of the forming device can be halted for example when the dimension of formed food products is out of range, and/or when foreign bodies are detected.

Dry Coater for Discrete Food Products

In a dry coater, discrete food products are coated with a dry coating. Such a station generally comprises a hopper for the dry coating, coating distribution means and possibly an air knife for blowing off excess dry coating.

An exemplary actuator of a dry coater is a distribution speed actuator for the dry coating, setting the coating rate. Other possible actuators are capable of setting a blow-off speed, a blow-off air temperature, and/or a food product transport rate.

Exemplary sensors of a dry coater monitor the dry coating consumption, and/or the stickiness of the food product prior to coating.

Exemplary controllable parameters in a dry coater include:
the blow-off air temperature and/or blow-off speed of the dry coater
the blow-off air temperature and/or blow-off speed of the dry
the coating rate

Wet Coating Device for Discrete Food Products

In a wet coating device, discrete food products are coated with a wet coating. Such a station optionally comprises a batter mixer to create the wet coating, a liquid vessel for the wet coating and distribution means.

Exemplary actuators of a wet coating device include actuators setting a blow-off speed, batter temperature, batter mixing speed, batter mixture, food product transport rate, dosing rate, etc.

Exemplary sensors provided in a wet coating device acquire data relating to the batter viscosity, batter temperature in batter mixer, batter temperature upon entry of coating device, wet coating consumption, etc.

Exemplary controllable parameters in a wet coating device include:
the blow-off air temperature and/or blow-off speed of the wet coating device
the blow-off air temperature and/or blow-off speed of the wet coating device

Fryer for Discrete Food Products

In a fryer, the discrete food products are fried. Commonly a deep fryer is applied in which the food products are submerged in hot fat, commonly oil. Other industrially applied frying techniques include a pressure fryer or vacuum fryer. In such a fryer station, optionally provisions are available for separating surplus frying fat, e.g. excess oil, from the fried discrete food products, e.g. by absorption or allowing the food products to drain out.

Exemplary actuators of a fryer include actuators setting the oil circulation, the oil composition, in particular the oil mixture old/new, etc. The dwell time in a fryer can e.g. be controlled by controlling a belt velocity on which food products are transported through the fryer.

Exemplary sensors provided in a fryer acquire data relating to oil quality, e.g. colour, oil filter information, oil consumption (per product), coating quality on food product upon leaving the fryer. Possibly a camera is provided to detect agglomerated products upon leaving the fryer. Also the temperature of the products are commonly measured, e.g. prior to and after the frying process. Such a sensor advantageously measures the core temperature of the products.

Exemplary controllable parameters in a fryer include:
the dwell time in the fryer
the dwell time and oil temperature in the fryer.

Heater for Discrete Food Products

In a heater the food products are subjected to a heat treatment, e.g. cooking the products. The heater optionally comprises a conveyor belt on which the discrete food products are transported. In embodiments, a convection heater is provided, e.g. with two climate chambers, which can be controlled individually.

Exemplary actuators of a heater include actuators setting the air circulation speed and/or a dew point.

Exemplary sensors of a heater are a dew point sensor and a camera detecting fatty (lecithin) imprints of the food products on the conveyor belt leaving the heater. Also, the temperature of the products are commonly measured, e.g. prior to and after the heating process. Such a sensor advantageously measures the core temperature of the products.

Exemplary controllable parameters in a heater include:
the dwell time and/or air circulation speed in the heater
the air temperature and/or air circulation speed and/or dwell time and/or dew point in the heater.

Freezer for Discrete Food Products

In a freezer, the discrete food products are frozen. Such a freezer commonly comprises a conveyor belt for transporting the food products, preferably creating Individual Quality Frozen (IQF) food products.

Exemplary sensors of a freezer include ambient temperature sensors inside and outside the freezer, and core temperature sensors to measure the core temperature of the products.

Exemplary controllable parameters in a freezer include:
the temperature in the freezer
the belt speed in the freezer to control the dwell time of the food products in the freezer.

The food processing line according to the present invention, comprises a processing line controller for controlling the food processing line. The processing line controller may be a single physical unit or may alternatively be distributed over several physical locations within the processing line. The processing line controller is configured to receive signals from the plurality of food processing stations and from said sensors. The processing line controller is further configured to send electronic signals to the plurality of food processing stations. The communication of these electronic signals is implemented in commonly known fashion, by means of appropriate electronic communication protocols. Communication may be implemented such that the process line controller is in direct communicative connection to a single component inside a food processing station or may alternatively be provided via a central interface component provided in a food processing station. The latter is commonly known, in particular in case the food processing line is composed of a plurality of food processing stations of a plurality of manufacturing companies, commonly using a specific communication protocol proprietary to such manufacturer.

The processing line controller according to the present invention comprises a data collection module for collecting sensor information, configured to receive sensor information from the at least one food product sensor, the at least one utility sensor and the at least one processing sensor, to store sensor information on a storage means and to communicate stored sensor information via an electronic communication line. Such data collection module may be integral part of the processing line controller or may alternatively be a separate module in communicative connection with the relevant components such as sensors, processing station interface components and/or the processing line controller. The data collection module is equipped with communication interfaces suitable for the available sensors and communication protocols to and from the plurality of food processing stations in an in itself known way of electronic communication. Storage of the sensor data is provided for by a database structure such as a relational and/or non-relational database. Such a database may be distributed and/or replicated over multiple physical locations either local within the production facility or remotely.

The processing line controller according to the present invention comprises input means for specifying at least one desired food product output characteristic. Such desired food product output characteristic is commonly defined in a food product specification and may include requirements on the range of weight distribution of the end products, its color, form, core temperature, surface temperature, etc. The input means may be implemented as a human machine interface such as a touch screen as integral part of the food processing line, but may also be provided in electronic form, e.g. as part of a database comprising a collection of food product specifications.

The processing line controller according to the present invention comprises input means for specifying a nominal operating condition for the utility supply station and for the processing station. The nominal operating condition is commonly determined during a food product development cycle, on the basis of e.g. product and/or process knowledge. The nominal operating condition is in some way comparable to a food product recipe, in which it is indicated how long, and under which conditions a food product needs to be processed. Based on the food product requirements and the specifics of the available food processing stations available in the food processing line, each nominal processing operation of the plurality of processing stations is determined. Nominal processing station settings such as e.g. pressure settings, temperature settings, humidity, air speeds, dwell times etc are stored and inputted via the input means for specifying a nominal operating condition. This input means may be provided as a human machine interface such as a touch screen as integral part of the food processing line but may also be provided in electronic form.

The processing line controller according to the present invention is a functional component of the food processing line and may be implemented as a single physical unit or distributed over multiple physical locations, either locally at the food production facility or remotely, such as at a remote networking location (“in the cloud”). The processing line controller comprises electronic components such as networking and other communication components, storage and memory components and at least one computer processor, such as a microprocessor. Functions of the production line controller may be implemented in electronic hardware components such as a PLC and/or may be implemented as software components that are executed on the at least one computer processor.

The processing line controller according to the present invention comprises an anomaly detection module configured to in operation detect an anomaly from the nominal operating condition based on the collected sensor information. In data science, an anomaly is defined as an observation which deviates so much from the other observations as to arouse suspicions that it was generated by a different mechanism. The anomaly detection module is communicatively connected to the data collection module and is able to read and/or retrieve sensor data that was collected and stored and comprises a processor, such as a microprocessor in order to process these collections of collected data points. The collected data is converted such that an anomaly detection algorithm that is executed in the microprocessor of the process line controller, is able to process the collected data or a subset thereof. By feeding food product sensor information, utility sensor information and processing sensor information into the anomaly detection module and applying anomaly detection algorithms, that are in itself known from (statistical) data science in general, it can be calculated what the probability is that the process, or a specific part or component is being operated under normal desired conditions or under anomalous conditions. As a result, the anomaly detection module is able to detect an anomaly earlier than known control mechanisms that compare a timestamped measurement with a reference value. Known data science anomaly detection algorithms that can be advantageously executed in the context of the present invention include statistical process control (SPC) or multivariate statistical process control (multivariate SPC). SPC and multivariate SPC can alternatively be applied more efficiently by applying principle component analysis (PCA) to compress the data that is analysed with SPC and/or multivariate SPC. These methods are known in itself in the field data science e.g. in “Statistical Process Control of Multivariate Processes” (J F. MacGregor, T. Kourti; Control Eng. Practise, Vol. 3, pp. 403-414, 1995). Other known data science anomaly detection algorithms that can be advantageously applied in the context of the present invention include machine learning algorithms, such as unsupervised machine learning algorithms to detect anomalous outlying data points. Suitable unsupervised machine learning anomaly detection algorithms that are in itself known in data science and can be advantageously applied in the context of the present invention, include the Isolation Forest algorithm, neural network auto-encoder algorithms, K-means clustering, Local Outlier Factor (LOF) algorithm etc. These algorithms typically will detect outlying data points based on the deviation from the probability distribution of the data.

The processing line controller according to the present invention comprises a root cause module configured to in operation determine a root cause of the detected anomaly using a statistical data analysis. The root cause module detection module is communicatively connected to the data collection module and is able to read and/or retrieve sensor data that was collected and stored and comprises a processor, such as a microprocessor in order to process these collections of collected data points. The root cause module is configured such that it is communicatively connected to the anomaly detection module such that a detected anomaly is communicated to the root cause module. The root cause module is executed in the microprocessor of the process line controller, is able to process the collected data or a subset thereof. The root cause module may be executed on the same computer processor as the anomaly detection module, on a separate computer processor or be integral part of an integrated suite of control modules that are executed on the processing line controller. The main aim of the root cause module is to try to determine the most likely root cause for any detected anomaly. The determination of the most likely root cause for a detected anomaly is implemented using a statistical data analysis. Suitable statistical data analysis algorithms that are in itself known from data science and can be advantageously applied to the present invention include supervised learning algorithms based on statistical data correlation analysis and/or a Failure Mode & Effect Analysis (FMEA) in which possible failure modes of the food processing line are collected such that future occurrences of such failure mode can be recognised by the root cause module. The supervised learning algorithms calculate the probability for each detected anomaly that the anomaly is caused by a specific root cause, such that the most probable root cause is determined.

The processing line controller according to the present invention comprises a corrective measure module configured to in operation determine a corrective measure in response to a detected anomaly and to provide the corrective measure to at least one physical actuator in the food processing line in order to control the food processing line such that the food product is processed in accordance with the desired food product output characteristic. The corrective measure module communicatively connected to the root cause module and is configured such that a determined root cause and optionally details of a detected anomaly is communicated to the corrective measure module. The corrective measure module is executed on a computer processor of the process line controller and is able to process data received from other modules. The corrective measure module may be executed on the same computer processor as the anomaly detection module and/or the root cause module, on a separate computer processor or be integral part of an integrated suite of control modules that are executed on the processing line controller. The main aim of the corrective measure module is to determine a corrective measure or a sequence of corrective measures that can be communicated to actuators within food processing stations in the food processing line and/or to actuators in utility supply stations, in order to change the operations of the food processing line such that the detected anomaly is counteracted, compensated, corrected and/or its influence on the end product is minimized. The corrective measure may also include a plan to stop a food processing station or even the complete food processing line in case of a catastrophic and disruptive event, e.g. at a utility supply station.

The food processing line according to the present invention, comprising a processing line controller using and processing said sensor information is able to act on the operating conditions of the food processing line right after an anomaly is detected. In many cases even before an end product that was processed by the food processing line during the anomaly is completely finished into an end product. Therefore, the amount of waste due to end product rejections and the processing resources wasted for rejected end products as a result of the end product not fulfilling the product specifications and/or requirements are significantly reduced compared with known processing line controllers, such as feedback and/or feedforward controllers that compare an end product with a certain threshold in order to change the processing for future products.

In an embodiment of the food processing line according to the present invention, the processing utility of the at least one utility supply station is one of the groups consisting of thermal oil, steam and pressurized air. Known food processing lines, commonly restrict control of individual processing stations to sensors within that specific processing station. A disturbance of a utility used in such processing station, such as thermal oil temperature, steam pressure and/or pressurized air may result in an end product further downstream the processing line at which moment in time it is detected. By using sensor information from the utility supply station, the processing line controller of the food processing line is able to correct for anomalies at the moment such a disturbance occurs instead of after the processing of a food product is completed in a processing station or even at the end of the complete processing line.

In an embodiment of the food processing line according to the present invention, at least one of the at least one food product sensor is configured to acquire one of the groups consisting of core temperature, surface temperature, weight, a product color, a product dimension and a product appearance characteristic. Such a sensor may measure all passing food products or take a selected food product at a certain regular interval. By acquiring a measure relating to any one of said food product properties the processing line controller is able to act in case one of the food product properties shows an anomaly. Preferably the food product sensor information comprises information relating to the desired food product output characteristics such that these properties can be tracked throughout the processing of the food product in the food processing line.

In an embodiment of the food processing line according to the present invention, the at least one processing sensor is configured to acquire one of the groups consisting of a climate characteristic at one of the plurality of processing stations and a dwell time of the product at one of the plurality of processing stations. This information enables the processing line controller to act in case of a detected anomaly, such as for example, a cabinet door opened by an operator or a faulty valve that could cause a utility such as steam or thermal oil (depending on the type of processing station) not entering the processing station in the desired nominal amount or condition.

In an embodiment of the food processing line according to the present invention, the processing line controller comprises an electronic actuator controller module for in operation controlling the at least one physical actuator in response to a corrective measure provided to the electronic actuator controller module. Suitable control algorithms for controlling such physical actuator that are in itself known from control technology and can be advantageously applied to the present invention include a linear controller algorithms such as a proportional differential controller (PD), a proportional integrating differential controller (PID), a model predictive controller (MPC), an LQG controller, fuzzy control algorithms, lookup tables (LUT), reinforcement learning algorithms such as deep deterministic policy gradient (DDPG), and logical automata controllers using if-then-logic. Said electronic actuator controller provides control signals to a single or a plurality of actuators in the food processing line based on the determined corrective measure at a specified moment in time and/or instantaneous. The electronic actuator controller module may be integral part of the processing line controller or alternatively be distributed over a plurality of physical locations, such as in any of the food processing stations.

In an embodiment of the food processing line according to the present invention, the processing line controller comprises a predictor module configured for determining in operation an estimated prediction of at least one food product output characteristic based on sensor information from the collection module, for example by applying sensor fusion methods. Suitable prediction algorithms for controlling such physical actuator that are in itself known from control technology and data science and can be advantageously applied to the present invention include a Kalman filter algorithm, a neural network algorithm and other common machine learning algorithms. The predictor module communicatively connected to the data collection module. The predictor module is executed on a computer processor of the process line controller and is able to process data received from other modules. The corrective measure module may be executed on the same computer processor as the anomaly detection module and/or the root cause module, on a separate computer processor or be integral part of an integrated suite of control modules that are executed on the processing line controller. The main aim of the predictor module is to provide an estimated prediction of at least one food product output characteristic based on sensor information, and optionally also based on measurements of past and current end products and optionally also based on control signals provided by the corrective measure module and/or the electronic actuator controller module. Such prediction may be advantageously used in the processing line controller to optimize corrective measures and to minimize the influence of anomalies on the end products of the food processing line.

In an embodiment of the food processing line according to the present invention, the root cause module comprises a supervised learning algorithm, wherein the detected anomaly in operation is labelled with a root cause label, using the collected sensor information of the data collection module and a labelling algorithm, preferably a failure mode & effect analysis (FMEA) labelling algorithm or a statistical data correlation analysis. By using a supervised learning algorithm the processing line controller can be trained to associate a current detected anomaly with a realistic root cause based on historic sensor data and expert knowledge from experts in the field of the processing stations and/or the food product development field.

In another aspect of the invention, a method for controlling a food processing line, the processing line comprising:
a plurality of physically separate processing stations in which a food product is subjected to one or more processing operations;
at least one utility supply station providing a processing utility to one or more processing stations;
a plurality of food product sensors configured to observe a food product condition;
at least one utility sensor configured to observe a utility condition;
at least one processing sensor configured to observe a processing station condition;
a processing line controller for controlling the food processing line, comprising a data collection module for collecting sensor information,
the method comprising the steps of
   A) providing at least one desired food product output characteristic to the processing line controller;
   B) providing a nominal operating condition for the utility supply station and for the processing station;
   C) collecting sensor information from the plurality of food product sensors and the at least one utility sensor and the at least one processing sensor into the data collection module;
   D) detecting an anomaly from the nominal operating condition by analysing the sensor information;
   E) determining a root cause of the anomaly;
   F) determining a corrective measure to correct for the anomaly;
   G) providing the corrective measure to at least one actuator in the food processing line in order to control the food processing line such that the food product is processed in accordance with the desired food product output characteristic.

In an embodiment of the method for controlling a food processing line according to the present invention, steps A and B are provided as an initial value before the food product is subjected to a processing operation in the food processing line. These steps may for example be performed at the installation of the food processing line, at the introduction of a new food product to be processed in the food processing line, at any modification of the sequence and/or configuration of the food processing line. In most use-cases steps A and B are not required to be executed continuously or for every batch of food product. Commonly the information provided in steps A and B are determined during a product development cycle and/or at the installation of new equipment such as a new food processing station.

In an embodiment of the method for controlling a food processing line according to the present invention, steps C and D are performed during the processing of the food product in the food processing line. In common use-cases, steps C and D are continuously executed in real-time during operation of the food processing line. Alternatively, steps C and D can be executed at regular or irregular intervals, for example one every second or triggered by the detection of a certain event in the food processing line, such as the detection of a food product at a certain location in the food processing line.

In an embodiment of the method for controlling a food processing line according to the present invention, steps E, F and G are executed in case an anomaly is detected in step D. Steps E, F and G may alternatively be implemented such that the steps are executed at each control cycle, whereas in absence of a detected anomaly the output is set to an appropriate value, such as zero, null or empty value.

In an embodiment of the method for controlling a food processing line according to the present invention, further comprising determining an estimated prediction of at least one predicted food product output characteristic, using the collected sensor information as input to a prediction algorithm and wherein the at least one predicted food product output characteristic relates to the at least one desired food product output characteristic as provided in step A. In a further embodiment the prediction algorithm comprises an algorithm from the group of Kalman filter, neural network and machine learning algorithm.

In an embodiment of the method for controlling a food processing line according to the present invention, wherein subsequent to step G, it further comprises the step of determining an electronic control signal, in response to the corrective measure of step G and providing the electronic control signal to at least one physical actuator in the food processing line.

In an embodiment of the method for controlling a food processing line according to the present invention, the electronic control signal is determined using a control algorithm based on at least one of linear PID-controller algorithm, model predictive controller algorithm, linear quadratic controller algorithm, and fuzzy control algorithm.

In an embodiment of the method for controlling a food processing line according to the present invention, step D utilizes a multivariate statistic control algorithm and/or an unsupervised machine learning algorithm.

In an embodiment of the method for controlling a food processing line according to the present invention, step E utilizes a supervised machine learning algorithm, wherein the detected anomaly is labelled with a root cause label, using the collected sensor information of the data collection module and a labelling algorithm.

In an embodiment of the method for controlling a food processing line according to the present invention, the labelling algorithm comprises a failure mode & effect analysis (FMEA) labelling algorithm or a statistical data correlation analysis.

Reference is made to details and advantages in the description of corresponding elements and functionality of the food processing line in the description here above. Further scope of applicability of the present invention will become apparent from the detailed description given hereinafter. However, it should be understood that the detailed description and specific examples, while indicating embodiments of the invention, are given by way of illustration only, since various changes and modifications within the scope of the invention will become apparent to those skilled in the art from this detailed description.

### BRIEF DESCRIPTION OF THE DRAWINGS

The present invention will become more fully understood from the detailed description given hereinbelow and the accompanying schematical drawings which are given by way of illustration only, and thus are not limitative of the present invention, and wherein:

FIG. 1 schematically illustrates a perspective drawing of a first embodiment of an inventive food processing line with five food processing stations,

FIG. 2 schematically represents a second embodiment of an inventive food processing line,

FIG. 3 schematically represents a third embodiment of an inventive food processing line,

FIG. 4 schematically represents an exemplary processing line controller architecture,

FIG. 5 schematically represents a fourth embodiment of an inventive food processing line,

FIG. 6A-C schematically demonstrates anomaly detection in an embodiment of an inventive food processing line,

### DETAILED DESCRIPTION OF EMBODIMENTS

The present invention will now be described with reference to the accompanying drawings, wherein the same reference numerals have been used to identify the same or similar elements throughout the several views.

It is noted that the drawings are schematic, not necessarily to scale and that details that are not required for understanding the present invention may have been omitted. The terms “upward”, “downward”, “below”, “above”, and the like relate to the embodiments as oriented in the drawings, unless otherwise specified. Further, elements that are at least substantially identical or that perform an at least substantially identical function are denoted by the same numeral, where helpful individualised with alphabetic suffixes.

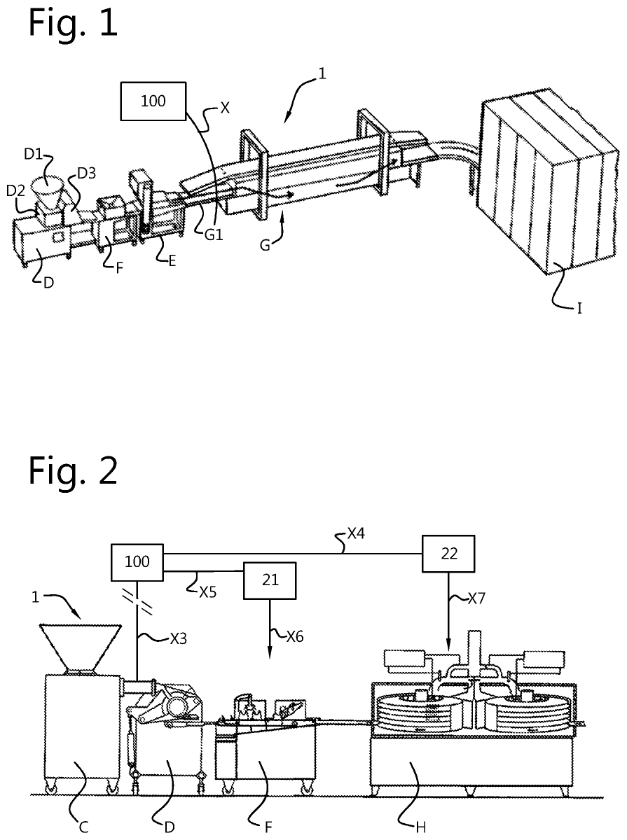

FIG. 1 schematically illustrates a perspective drawing of a first embodiment of an inventive food processing line with five food processing stations. In FIG. 1 a first embodiment of an inventive food processing line 1 is shown, for in-line processing food. The shown food process line 1 comprises five processing stations. In succession:
a forming station D, here a moulding device for moulding three-dimensional discrete food products from a mass. In particular, the moulding device D is designed to produce discrete food products from a mass of pounded meat, for example hamburgers or nuggets;
a wet coating device F, here a device designed to coat the outside of discrete food products with a layer of a liquid material, e.g. batter;
a dry coater E, here a crumbing device which can be used to apply a layer of coating material in crumb form to the outside of discrete food products;
a fryer G, here provided with a deep-frying bath;
and a freezer I for freezing discrete food products.

The forming station D comprises a hopper D1, a pump D2 and a mould drum D3. The pump D2 preferably comprises a sensor acquiring data relating to the food mass, such as temperature and viscosity. The shown fryer G comprises a conveyor G1, e.g. a belt conveyor, transporting the food products through the fryer. Advantageously, the velocity of the conveyor can be controlled, to adjust the dwell time of the food products in the fryer.

The pump D2 of the forming station D is provided with a sensor, and the fryer G is provided with an actuator, here conveyor G1. A processing line controller 100 is provided, which is communicatively connected to the actuators and sensors of the system. In this schematic drawing, communication line x communicatively connects processing line controller 100 with the processing line's sensors and actuators. In this example, all processing stations are physically connected with each other either directly or by means of a conveyor belt, as shown between fryer G and quick freezer I. Besides the physical connection, the processing line stations are further connected by means of interstation communication by means of an electronic communication means, in this example each processing stations are connected by means of interface cards that are connected by a cable. A central communication interface connects this interstation communication line with the processing line controller 100 with connection x, such that the processing line controller 100 is communicatively connected to all sensors and actuators of the processing line. The processing line controller is further connected to sensors and actuators at a remote utility station (not shown). In this case it is connected to a thermal oil heater, which is located in another building in this food processing facility. This thermal oil heater heats thermal oil with a gas burner and provides the hot thermal oil to the fryer G in this food processing line 1, but also to another remote food processing line (not shown) which processes a different food product.

FIG. 2 schematically represents a second embodiment of an inventive food processing line, wherein the processing line comprises three processing stations. In succession:
a forming station D, here a moulding device for moulding three-dimensional discrete food products from a mass. In particular, the moulding device D is designed to produce discrete food products from a mass of pounded meat, for example hamburgers or nuggets. Preferably, a camera D1 is provided downstream of the moulding device, detecting the food product dimensions upon leaving the forming station D;
a wet coating device F, here a device designed to coat the outside of discrete food products with a layer of a liquid material, e.g. batter;
a heater H, here an oven for heating discrete food products. Preferably, the heater H comprises actuators of such as the air temperature and/or air circulation speed and/or dwell time in the convection heater.

According to the invention, an interstation processing line controller 100 is provided, communicatively connected with all sensors available in the food processing line including the camera D1 of the forming station and all controllable actuators. This communicative connection is implemented as communication line x3 between the processing line 1 and the processing line controller 100. The controller 100 is further connected to all available sensors of the utility stations 21 and 22 by means of communication lines x5 and x4 respectively. Utility station 21 provides pressurized air via pressurized air tube x6 to both the wet coater station F to blow off excess coating and to the forming station D in order to release formed products from the moulds. The food processing facility has a steam boiler 22 in a separate building which provides steam to several processing stations in the food processing facility. Steam pipe x7 provides steam under high pressure from the steam boiler 22 utility station to the heater station H which is in this exemplary embodiment a double spiraled multi climate modular oven system. In the oven system H, the dwell time can be controlled by setting the conveyor belt speed, dew point can be controlled by the valve at the end of steam pipe x7 and the temperature is controlled by an internal heater element which is servo controlled by means of several thermocouple sensors inside of the modular oven. The control setpoint of the oven for all of the controllable properties can be set by process line controller 100 which is connected to the processing line 1 via communication line x3. Communication lines x3, x4 and x5 are implemented as wireless connections in this innovative processing line according to the invention. Whereas the interstation communication is implemented as wired communication lines with interfacing cables, the communication between the controller and the processing line and between the supply stations and the processing line controller are wireless. The communication is performed by in itself known wireless communication means and protocols.

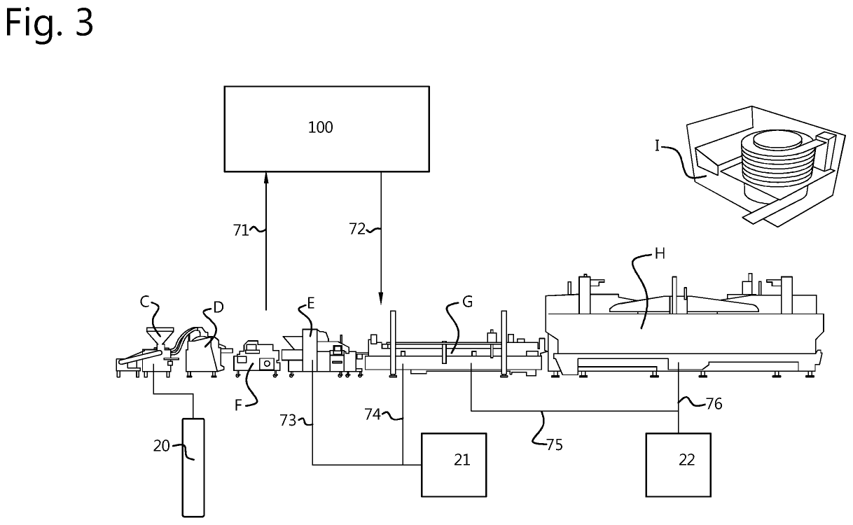

FIG. 3 schematically represents a third embodiment of an inventive food processing line, wherein the processing line comprises three processing stations. In succession:
a food preparation station C, mincing pieces of meat while including and mixing marinades and seasoning in accordance with the food recipe of current food product. Utility station 20 comprises a supply for carbon dioxide (CO2) as is commonly added into the mixture. At the connection of the CO2 supply is a valve with an integrated pressure sensor, which measures the pressure of the carbon dioxide at the supply and sends its measurements to processing line controller 100 in a wireless fashion.
a forming station D, here a moulding device for moulding three-dimensional discrete food products from a mass. In particular, the moulding device D is designed to produce discrete food products from a mass of pounded meat, for example hamburgers or nuggets. Preferably, a camera D1 is provided downstream of the moulding device, detecting the food product dimensions upon leaving the forming station D;
a wet coating device F, here a device designed to coat the outside of discrete food products with a layer of a liquid material, e.g. batter;
a dry coater E, here a device designed to coat the outside of the discrete food products with a layer of dry coating material, such as e.g. crumbs, breading, panko etc.
a fryer station G, here provided with a deep-frying bath;
a heater H, here an oven for heating discrete food products. Preferably, the heater H comprises actuators of such as the air temperature and/or air circulation speed and/or dwell time in the convection heater.
a freezer I for freezing discrete food products.

The food processing stations C, D, F, E, G and H are physically connected directly in that the output side of a first station is placed adjacent to the input side of the next station such that food products are conveyed through all subsequent stations. The quick freezer I is in this embodiment located relatively remotely, in that no conveyor belt is available between the oven and the freezer. In this embodiment products from the oven are placed on a trolley rack (not shown). When the trolley rack is filled with products, the trolley rack is rolled to the freezer I for further processing, in this case freezing of the products. It will be appreciated by the skilled person that depending on the configuration of the food processing facility and the specifics of the food processing line 1, stations may be directly connected, connected by a conveyor belt and/or connected by a batch-wise operator conveyor, such as the trolley rack in this embodiment. The processing line 1 further comprises a thermal oil heater utility station 22 heating thermal oil which is supplied to the fryer station G and the heater H via supply lines 75 and 76, and utility station 21 in this example providing pressurized air to the dry coater and to the entrance of the fryer G for blowing of loose coating material via supply lines 74 and 73. It will be appreciated by the skilled person that utilities may be physically close to the processing line 1 or located remotely, such as e.g. in a separate building, on the roof of a building or in a different area/room of the production building.

According to the invention, an interstation processing line controller 100 is provided, communicatively connected with available sensors and actuators in the food processing line, including sensors and actuators in the utility supply stations, schematically indicated by arrows 71 and 72 respectively. These connections 71 and 72 are schematically depicted as individual lines but may in practise by a single combined bi-directional communication line and/or may alternatively be implemented as a large number of individual communication lines, either wired, wireless or combinations thereof.

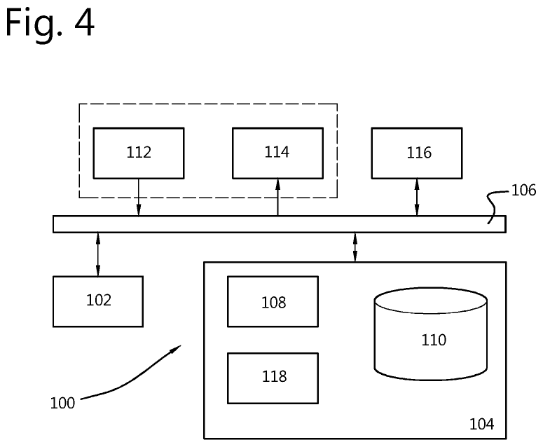

FIG. 4 schematically represents an exemplary processing line controller architecture, illustrating an exemplary data processing system that may be used in a computing system as described throughout this application in the processing line controller, but also at local processing stations for local machine control and/or interstation communication and/or communication between processing stations and the process line controller.

As shown in FIG. 4, the processing line controller 100 may include at least one processor 102 coupled to memory elements 104 through a system bus 106. As such, the data processing system may store program code within memory elements 104. Further, the processor 102 may execute the program code accessed from the memory elements 104 via a system bus 106. In one aspect, the data processing system may be implemented as a computer that is suitable for storing and/or executing program code. It should be appreciated, however, that the processing line controller 100 may be implemented in the form of any system including a processor and a memory that is capable of performing the functions described within this specification.

The memory elements 104 may include one or more physical memory devices such as, for example, local memory 108 and one or more bulk storage devices 110. The local memory may refer to random access memory or other non-persistent memory device(s) generally used during actual execution of the program code. A bulk storage device may be implemented as a hard drive or other persistent data storage device. The processing system 100 may also include one or more cache memories (not shown) that provide temporary storage of at least some program code in order to reduce the number of times program code must be retrieved from the bulk storage device 110 during execution.

Input/output (I/O) devices depicted as an input device 112 and an output device 114 optionally can be coupled to the data processing system. Examples of input devices may include, but are not limited to, a keyboard, a pointing device such as a mouse, or the like. Examples of output devices may include, but are not limited to, a monitor or a display, speakers, or the like. Input and/or output devices may be coupled to the data processing system either directly or through intervening I/O controllers.

In an embodiment, the input and the output devices may be implemented as a combined input/output device (illustrated in FIG. 4 with a dashed line surrounding the input device 112 and the output device 114). An example of such a combined device is a touch sensitive display, also sometimes referred to as a “touch screen display” or simply “touch screen”. In such an embodiment, input to the device may be provided by a movement of a physical object, such as e.g. a stylus or a finger of a user, on or near the touch screen display.

A network adapter 116 may also be coupled to the data processing system to enable it to become coupled to other systems, computer systems, remote network devices, and/or remote storage devices through intervening private or public networks. The network adapter may comprise a data receiver for receiving data that is transmitted by said systems, devices and/or networks to the processing line controller 100, and a data transmitter for transmitting data from the processing line controller 100 to said systems, devices and/or networks. Modems, cable modems, and Ethernet cards are examples of different types of network adapter that may be used with the processing line controller 100.

As pictured in FIG. 4, the memory elements 104 may store an application 118. In various embodiments, the application 118 may be stored in the local memory 108, the one or more bulk storage devices 110, or apart from the local memory and the bulk storage devices. It should be appreciated that the processing line controller 100 may further execute an operating system (not shown in FIG. 4) that can facilitate execution of the application 118. The application 118, being implemented in the form of executable program code, can be executed by the processing line controller 100, e.g., by the processor 102. Responsive to executing the application, the data processing system 100 may be configured to perform one or more operations or method steps described herein.

In yet another aspect, the processing line controller 100 may be distributed over several physical units and comprise a server component. For example, the processing line controller may represent an (HTTP) server, in which case the application 118, when executed, may configure the data processing system to perform (HTTP) server operations.

Various embodiments of the invention may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored. The computer program may be run on the processor 102 described herein.

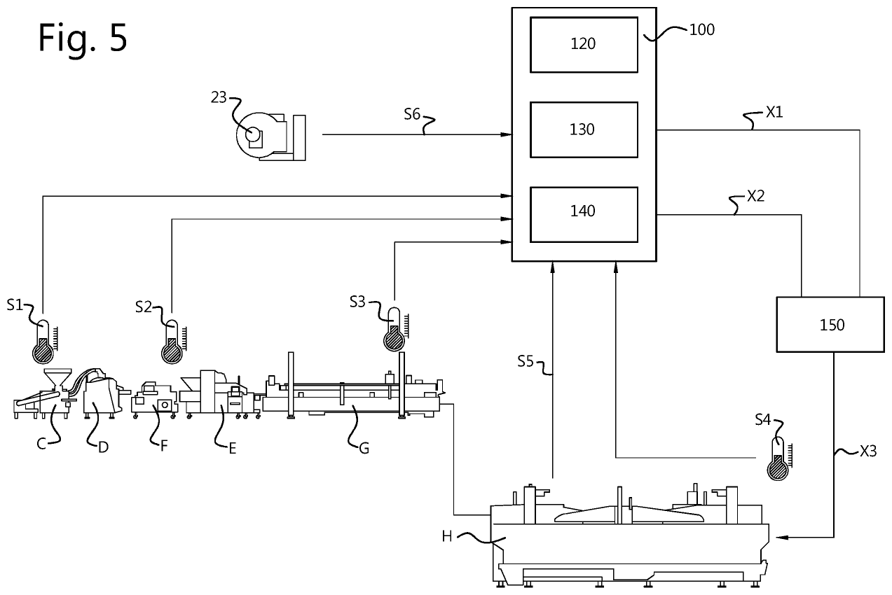

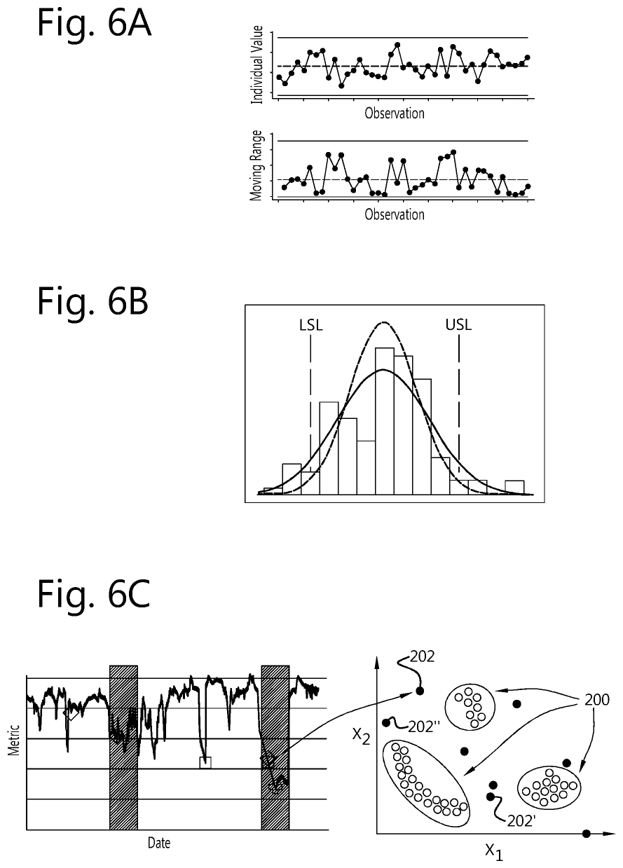

The inventive process line controller 100 according to the invention and its interaction with the food processing line 1 as applied in all previous embodiments will hereinafter be further discussed in more detail with reference to the schematically depicted processing line controller 100 of FIG. 5 and the illustrations of FIGS. 6A-C combined. FIG. 5 schematically represents an exemplary configuration of the innovative processing line according to the invention and comprises seven processing stations. In succession:
a food preparation station C, mincing pieces of meat while including and mixing marinades and seasoning in accordance with the food recipe of current food product. Utility station 20 comprises a supply for carbon dioxide (CO2) as is commonly added into the mixture. At the connection of the CO2 supply is a valve with an integrated pressure sensor, which measures the pressure of the carbon dioxide at the supply and sends its measurements to processing line controller 100 in a wireless fashion.
a forming station D, here a moulding device for moulding three-dimensional discrete food products from a mass. In particular, the moulding device D is designed to produce discrete food products from a mass of pounded meat, for example hamburgers or nuggets. Preferably, a camera D1 is provided downstream of the moulding device, detecting the food product dimensions upon leaving the forming station D;
a wet coating device F, here a device designed to coat the outside of discrete food products with a layer of a liquid material, e.g. batter;
a dry coater E, here a device designed to coat the outside of the discrete food products with a layer of dry coating material, such as e.g. crumbs, breading, panko etc.
a fryer station G, here provided with a deep-frying bath;
a heater H, here an oven for heating discrete food products. Preferably, the heater H comprises actuators of such as the air temperature and/or air circulation speed and/or dwell time in the convection heater.
a freezer (not shown) for freezing discrete food products.

The food processing stations C, D, F, E, G and H are physically connected directly in that the output side of a first station is placed adjacent to the input side of the next station such that food products are conveyed through all subsequent stations.

The processing line 1 further comprises several utility supply stations like a thermal oil heater, a steam boiler, compressed air supply stations and a carbon dioxide supply station, all schematically indicated by utility supply station 23, which is communicatively connected with the process line controller 100 via communication line S6. It will be clear to a skilled person that communication line S6 is illustrative for any of the previously discussed communication configurations, both wired as wireless.

Throughout the processing line 1 a plurality of sensors have been mounted such as temperature sensor S1 for measuring the meat mass in meat preparation station C, food product surface temperature sensor S2 mounted at the entrance area of the fryer G, temperature sensor S3 mounted in the thermal frying oil to measure the frying oil temperature, core temperature sensor S4 at the output area of the modular oven H to measure the core temperature of food products leaving the oven and visual food product inspection camera S5 to inspect the product surface characteristics of the food products such as surface color, product dimensions, colorations, specks detection etc. These sensors S1-S5 are a couple of exemplary sensors for the wide range of sensors available throughout the food processing line 1. Other sensors are available in the processing line but have been omitted in the drawing for reasons of readability. These sensors can be categorized as food product sensor, utility sensor and processing sensor.

Food product sensors measure a property of the food products. Examples of food product sensors are e.g. food surface temperature sensor, food core temperature sensor, meat mass temperature sensor, weight sensor for measuring the weight of the food products, camera system/sensor for acquiring measurements on surface color, product dimensions, (de)colorations, specks detection, undesired product marriages (two or more discrete products sticking together), etc.

Utility sensors acquire a utility condition measure. Utilities are auxiliary resources that aid in the processing of the food products but are not core components of the food product, such as meat dough, coating material etc. Note however that remnants of utilities may be found in the end product, such as thermal oil when fried and/or gasses that were used to improve product structures. Examples of utility sensors are e.g. temperature sensors in the thermal oil heater, flow sensor for acquiring a measurement on the flow of fluids like thermal oil, water, gasses, pressure sensors for measuring the pressure of compressed air and/or other fluids, etc.

Processing sensors are sensors that acquire a processing station condition measure inside or at a processing station.

Examples of processing sensors are temperature sensors inside the oven, dew point sensors inside the oven, electric power sensors for measuring the amount of power used by electric heaters and/or electric motors in the processing stations.

The processing line controller 100 controls the processing line. The processing line controller has a hardware architecture as described in relation to FIG. 4 and executes software code that functionally performs specific tasks within the processing line. The software code itself may be a unitary piece of software, or alternatively be split up into several individually executed pieces of code. For reasons of readability, the description of the processing line controller is split up into functional modules.

The data collection module collects all received sensor information of sensors that as communicatively connected to the processing line controller. The data collection module collects sensor information from food product sensors, utility sensors and processing sensors. The sensor data is received via one or more interface components and comprise low level data acquisition components for signals that require so in order to be processed. Some sensors send their information at intervals, some sensors need a request from the data collection module in order to communicate their information with the controller. The data collection module is equipped for any of these data receiving mechanisms. The received sensor data is stored on a storage means, in this example a database that is physically located as a hardware component inside of the processing line controller. Alternatively, the data base may also be located remotely, such as e.g. in the cloud or at a specified network location. The data collection module is configured to communicate the stored data or a specified subset thereof to other functional modules of the processing line controller upon request or at specified intervals, as will be illustrated hereafter.

The processing line controller has input means for specifying desired food product output characteristics, the so-called product specifications. These are commonly specified by the customer of the processing line or by the food processing facilities management. These specifications typically comprise requirements that the end products need to fulfil such as weight per product, fat content, surface color, but also food safety related properties such as core temperature after cooking and/or frying and core temperature after freezing. These specifications are inputted by the operator before starting operations. Typically these product specifications are determined and inputted in a database and read by the processing line controller when an operator indicates the product code for a food product to be processed from that moment. Many of the processing lines according to the invention are flexible lines that can be configured to produce several types of food products.

The processing line controller 100 has input means for specifying a nominal operating condition for the utility supply station(s) and for the processing stations. During the development cycle of a food product, the development team determines how to process a specific food product on the processing line available in the facility. Properties like meat mass temperature, amount of seasoning, pressure of the steam supply, temperature of the thermal oil from the thermal oil heater, fill pressure of the forming station, amount of coating, conveyor speed, fryer temperature and dwell time, temperature, dew point, air speed and dwell time in the oven etc are all investigated and determined. All of these nominal operating conditions are inputted into the processing line controller. Again, this input is typically done before starting the production of such a product and read by the processing line controller from a database in response to inputting the product code indicating which product will be processed. Alternatively, all of these settings may be inputted manually at the time of processing start by means of a keyboard and/or touch screen or the like.

The processing line controller comprises an anomaly detection module 120. This anomaly detection module 120 receives and/or reads sensor data from the data collection module and processes the sensor data by means of an anomaly detection algorithm. In this embodiment the anomaly detection module uses a statistical process control (SPC) algorithm, but other anomaly detection algorithms from data science, such as an unsupervised machine learning algorithm are alternatively available for performing a statistical anomaly detection analysis. The processing line controller 100 according to the invention receives a sequence of measurements from the sensors mentioned hereabove and applies a statistical data analysis to these measurements in order to determine whether an anomaly occurred or whether the process within the nominal operating conditions. As shown in FIG. 6A measurements from sensors enter the anomaly detection module as a sequence of time stamped measurements. Many processes and subprocesses generate characteristics that can have a certain distribution such as depicted in FIG. 6B. Such normal distribution is commonly known in data science and industrial processes. Such distribution is assumed for the product characteristics, but also for process conditions and supply station conditions. Food product specifications are typically defined by means of a lower specification limit (LSL) and an upper specification limit (USL), in that the desired weight of an end product when exiting the modular oven is set and a specified number of products is allowed to be lower and a specified number of products is allowed to be higher, as indicated and defined by the LSL and USL. Such distribution can also be defined for core temperature, number of specks on the surface of a food product etc. The anomaly detection module receives measurements of the sensors in the processing line as depicted in FIG. 6C on the left-hand side. From a time-based sequence of measurements it can be very difficult to determine when an anomaly occurs in that the processing line controller should control one of more actuators in the processing line to correct for the occurred anomaly. Instead of analysing the time-based sequence of measurements, the anomaly detection module applies SPC or to be more specific a multivariate version of SPC to the received measurements and determines data clusters 200 as shown in FIG. 6C on the right-hand side. Data clusters 200 are data points result from the time-based measurements as received and belong to a data cluster 200 if they are in close proximity in the dimensional space of the (multivariate) SPC processing, an anomaly point 202, 202′, 202″ would be a data point that lays remote from such data cluster 200. FIG. 6C shows an illustrative two-dimensional space spanned by theoretical characteristics x1 and x2. In a real world SPC analysis in an anomaly detection module, typically a more dimensional SPC space would be used, such as a data point synthesized from the heater power setpoints for the oil supply heater, oven and fryer, the measured oil temperature at the oil entrance of the fryer, the measured oil temperature at the oil exit of the fryer, the oil flow measured in the fryer, measured air speed inside of the oven, the valve settings for steam supply and the oil supply, the conveyor speed and the core temperature of the food product measured at the exit of the oven. This 11-dimensional data point is processed by the anomaly detector, and it is determined whether the current processing conditions are within nominal operating conditions or should be considered an anomalous data point. In case the anomaly detection module determines that the data point should be considered an anomalous data point, because it lays remote from the current data clusters 200 in the 11-dimensional analysis space, a signal is transmitted to the processing line controller 100.

In case the anomaly detection module 120 detects an anomalous data point, the root cause module 130 determines the root cause of the detected anomaly using another statistical data analysis. The root cause module 130 comprises a supervised learning algorithm, wherein the detected anomaly in operation is labelled with a root cause label, using the collected sensor information of the data collection module and a labelling algorithm, such as a failure mode & effect analysis (FMEA) labelling algorithm. Alternatively, the labelling algorithm uses a statistical data correlation analysis in order to determine the root cause of an anomaly when such is detected. For example, when an anomaly is detected in a system as described with reference to FIG. 5, the anomalous data point is analysed by the root cause module as one or more of the measured conditions does not fulfil the nominal operation conditions. The root cause module analyses the data from the data collection module, including the sensor data, but also the data relating to current actuator settings and calculates the most probable root causes for the detected anomaly. Such analysis can for example result in a calculation indication that the anomaly is caused by any one of the following causes; too cold meat in meat preparation station C, to thick coating as applied in coating stations F and E, two products sticking together before the fryer G, thermal oil heater is not getting enough gas supply to heat the thermal oil etc. All of these causes will be accompanied with a probability score based on the collected data. In this example, the pressure sensor of the gas supply to the thermal oil heater is indicated as 99.2% probability that this is causing the anomaly. Other indications include product dimensions measured too big 20.3%, meat supply in meat preparation station is too cold 34%, etc. The root cause module is configured to communicate the calculated probabilities of the possible root causes to other modules of the processing line controller such as the corrective measure module.

The processing line controller comprises a corrective measure module 140 which uses the outcome of the root cause module in order to determine a plan to correct the processing line such that the processing line is able to perform within its nominal operating conditions and/or to compensate for the root cause(s). The corrective measure module gets the input from the root cause module and determines a sequence of actions to be taken. In this example the gas valve to the gas supply for the thermal oil heater will be actuated from 60% open to 90% open to compensate for the reduced pressure as a first action to be taken immediately, further an alarm is sent to an operator in that the gas supply needs maintenance, thirdly the conveyor belt through the fryer is set to 90% of its current speed in order to compensate for the slightly reduced oil temperature effective immediately. All in all, this sequence of actions is executed such that the end products will continue to be within the limits of the set food product specifications.

In order to physically control actuators in the processing line, the processing line comprises an electronic actuator controller module 150 for in operation controlling the at least one physical actuator in response to a corrective measure provided to the electronic actuator controller module 150. The electronic actuator controller module translates the determined sequence of actions from the corrective measure module 140 into control signals to specific actuators, such as a sequence of pulses to a stepper motor for changing a valve setting, or a control voltage to an electric motor driving the conveyor belt. The control signals are communicated via communication line x3. In this example a fuzzy logic controller is applied to control the speed of the conveyor belt and a PID-controller algorithm is used for controlling the gas valve. Advantageous control algorithms applied for other actuators include linear PID-controllers, a model predictive controller, a linear quadratic controller, and a fuzzy controller.

The electronic actuator controller module 150 is supplied with the corrective measure module output and is further supplied with the output from a predictor module (not shown) configured for determining in operation an estimated prediction of at least one food product output characteristic based on sensor information from the collection module. In this example a Kalman filter is implemented in order to predict the end product core temperature behind the oven. The Kalman filter is fed with sensor information, current measured core temperatures and control signals, in order to predict the influence of the current state of the processing line on the output characteristics of the food product. Alternatively, the prediction module comprises a neural network and/or a machine learning algorithm in order to calculate these predictions. The predicted values are fed to the controller module 150 via communication line x2, whereas the output of the corrective measure module is inputted into the controller module 150 via communication line x1.

Various embodiments may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored.

Detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. In particular, features presented and described in separate dependent claims may be applied in combination and any advantageous combination of such claims are herewith disclosed.

Further, the terms and phrases used herein are not intended to be limiting; but rather, to provide an understandable description of the invention. The terms “a” or “an”, as used herein, are defined as one or more than one. The term plurality, as used herein, is defined as two or more than two. A plurality may also indicate a subset of two or more, out of a larger multitude of items. The term another, as used herein, is defined as at least a second or more. The terms including and/or having, as used herein, are defined as comprising (i.e., open language). The term coupled, as used herein, is defined as connected, although not necessarily directly.

Elements and aspects discussed for or in relation with a particular embodiment may be suitably combined with elements and aspects of other embodiments, unless explicitly stated otherwise. The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1.-20. (canceled)

21. A food processing line for processing a food product, comprising:
a plurality of processing stations in which the food product is subjected to one or more processing operations;
at least one utility supply station providing a processing utility to one or more of the processing stations;
at least one food product sensor configured to acquire a food product condition measure;
at least one utility sensor configured to acquire a utility condition measure;
at least one processing sensor configured to acquire a processing station condition measure;
a processing line controller for controlling the food processing line, comprising:
a data collection module for collecting sensor information, configured to:
receive sensor information from the at least one food product sensor, the at least one utility sensor and the at least one processing sensor;
store sensor information on a storage means;
communicate stored sensor information via an electronic communication line;
input means for specifying at least one desired food product output characteristic;
input means for specifying a nominal operating condition for the utility supply station and for the processing station;
an anomaly detection module configured to in operation detect an anomaly from the nominal operating condition based on the collected sensor information;
a root cause module configured to in operation determine a root cause of the detected anomaly using a statistical data analysis;
a corrective measure module configured to in operation determine a corrective measure in response to a detected anomaly and to provide the corrective measure to at least one physical actuator in the food processing line in order to control the food processing line such that the food product is processed in accordance with the desired food product output characteristic.

22. The food processing line according to claim 21, wherein the processing utility of the at least one utility supply station is one of the groups consisting of thermal oil, steam and pressurized air.

23. The food processing line according to claim 21, wherein at least one of the at least one food product sensor is configured to acquire one of the groups consisting of core temperature, surface temperature, weight, a product color, a product dimension and a product appearance characteristic.

24. The food processing line according to claim 21, wherein the at least one processing sensor is configured to acquire one of the groups consisting of a climate characteristic at one of the pluralities of processing stations and a dwell time of the product at one of the plurality of processing stations.

25. The food processing line according to claim 21, wherein the processing line controller comprises an electronic actuator controller module for in operation controlling the at least one physical actuator in response to a corrective measure provided to the electronic actuator controller module.

26. The food processing line according to claim 25, wherein the processing line controller comprises a predictor module configured for determining in operation an estimated prediction of at least one food product output characteristic based on sensor information from the collection module.

27. The food processing line according to claim 26, wherein the estimated prediction of at least one food product output characteristic from the prediction module relates to the at least one desired food product output characteristic.

28. The food processing line according to claim 21, wherein the anomaly detection module comprises a multivariate statistic process control algorithm and/or an unsupervised machine learning algorithm.

29. The food processing line according to claim 21, wherein the root cause module comprises a supervised learning algorithm,
wherein the detected anomaly in operation is labelled with a root cause label, using the collected sensor information of the data collection module and a labelling algorithm, a failure mode & effect analysis (FMEA) labelling algorithm or a statistical data correlation analysis.

30. A method for controlling a food processing line, the processing line comprising:
a plurality of physically separate processing stations in which a food product is subjected to one or more processing operations;
at least one utility supply station providing a processing utility to one or more processing stations;
a plurality of food product sensors configured to observe a food product condition;
at least one utility sensor configured to observe a utility condition;
at least one processing sensor configured to observe a processing station condition;
a processing line controller for controlling the food processing line, comprising a data collection module for collecting sensor information,
the method comprising the steps of:
A) providing at least one desired food product output characteristic to the processing line controller;
B) providing a nominal operating condition for the utility supply station and for the processing station;
C) collecting sensor information from the plurality of food product sensors and the at least one utility sensor and the at least one processing sensor into the data collection module;
D) detecting an anomaly from the nominal operating condition by analysing the sensor information;
E) determining a root cause of the anomaly;
F) determining a corrective measure to correct for the anomaly;
G) providing the corrective measure to at least one actuator in the food processing line in order to control the food processing line such that the food product is processed in accordance with the desired food product output characteristic.

31. The method according to 30, wherein steps A and B are provided as an initial value before the food product is subjected to a processing operation in the food processing line.

32. The method according to claim 20, wherein steps C and D are performed during the processing of the food product in the food processing line.

33. The method according to claim 30, wherein steps E, F and G are executed in case an anomaly is detected in step D.

34. The method according to claim 30, further comprising determining an estimated prediction of at least one predicted food product output characteristic, using the collected sensor information as input to a prediction algorithm and
wherein the at least one predicted food product output characteristic relates to the at least one desired food product output characteristic as provided in step A.

35. The method according to 34, wherein the prediction algorithm comprises an algorithm from the group of Kalman filter, neural network and machine learning algorithm.

36. The method according to claim 30, subsequent to step G, further comprising the step of determining an electronic control signal, in response to the corrective measure of step G and providing the electronic control signal to at least one physical actuator in the food processing line.

37. The method according to claim 36, wherein the electronic control signal is determined using a control algorithm based on at least one of linear PID-controller, model predictive controller, linear quadratic controller, and fuzzy controller.

38. The method according to claim 30, wherein step D utilizes a multivariate statistic control algorithm and/or an unsupervised machine learning algorithm.

39. The method according to claim 30, wherein step E utilizes a supervised machine learning algorithm, wherein the detected anomaly is labelled with a root cause label, using the collected sensor information of the data collection module and a labelling algorithm.

40. The method according to claim 39, wherein the labelling algorithm comprises a failure mode & effect analysis (FMEA) labelling algorithm or a statistical data correlation analysis.
